(19) United States
(12) Patent Application Publication
(10) Pub. No.: US 2026/0199053 A1
(43) Pub. Date: Jul. 16, 2026

(54) GLASS-TAG FIXATION FOR MEDICAL INSTRUMENTS

(71) Applicant: Aesculap AG, Tuttlingen (DE)

(72) Inventors: Ralf Pfister, Trossingen (DE); Andre Buerk, Villingen-Schwenningen (DE); Martin Machill, Rietheim-Weilheim (DE); Maximilian Bieber, Filderstadt (DE)

(21) Appl. No.: 19/134,738

(22) PCT Filed: Dec. 1, 2023
(86) PCT No.: PCT/EP2023/083911

§ 371 Date: Jun. 1, 2025

(30) Foreign Application Priority Data
Dec. 2, 2022 (DE) 10 2022 132 032.4

(51) Int. Cl. A61B 90/98; G06K 19/077

(52) U.S. Cl. CPC A61B 90/98; G06K 19/077

(57) ABSTRACT

A protective housing accommodates a transponder. The protective housing can be accommodated in a transponder receptacle in a medical product, for example in a medical instrument and/or medical product in a sterilization circuit. The protective housing can include a protective housing groove for accommodating a connecting spring.

### CROSS-REFERENCE TO RELATED APPLICATIONS

This application is the United States national stage entry of International Application No. PCT/EP2023/083911, filed on Dec. 1, 2023, and claims priority to German Application No. 10 2022 132 032.4, filed on Dec. 2, 2022. The contents of International Application No. PCT/EP2023/083911 and German Application No. 10 2022 132 032.4 are incorporated by reference herein in their entireties.

### FIELD

The present disclosure relates to a protective housing for a glass tag, a medical product, a connecting spring, a protective housing system and a medical product system.

### BACKGROUND

Since life cycle management of sterile goods is becoming more and more important in the future, the requirements for monitoring and trackability of these sterile goods, in particular of medical/surgical instruments in a sterilization cycle, are increasing. For this reason, medical instruments of all kinds are equipped with transponders, in particular passive transponders of RFID (radio-frequency identification) or NFC (near field communication) technology. This means that the location of the medical instruments can always be identified and a statement can be made as to whether they have (correctly) passed through the entire reprocessing process or all maintenance steps. It is also possible to detect the number of sterilization cycles a sterile item has undergone and to determine when the maximum service life of the sterile item has been reached. The configuration of the transponder in the form of a glass tag is particularly advantageous.

Devices for attaching transponders to medical instruments are already known from the prior art. For example, in document EP 2 087 850, a transponder is attached to the medical instrument, for example to its handle part, using a housing, wherein the housing has a cavity for receiving the transponder. The housing may consist of two separate parts which enclose the transponder and the handle part of the instrument in a notch during a fastening process and can then be connected in a form-fitting manner. Another fastening option is to place the transponder in a protective housing, which is foldable via a hinge and which is then strapped to the medical instrument using a flexible strap/belt.

A metallic fixing element for metallic RFID tags is known from document EP 3 146 477. The fixing element is attached/glued to a medical instrument via a flat base surface and holds the transponder via a notch enclosed by side surfaces. The transponder and the fixing element are electrically conductively connected to each other, which can improve the signal strength and range of the transponder, in particular when it is attached to smaller objects.

Due to the attachment of the transponder to the outside of a medical object and the resulting protrusions, attaching the transponder as described above affects the handling and ergonomics of the equipped sterile goods and may also make medical reprocessing or cleaning more difficult.

In general, there are also ways known of attaching a transponder to or in a medical instrument housing by gluing/potting/welding or in previously drilled holes. However, these solutions are complex and require appropriate preparations, such as ensuring that the surface of the medical instruments is clean and free of grease. In addition, pressing in via force-fit or gluing the transponder is not permanently resistant due to plastic setting. Furthermore, such a connection of the transponder to the instrument makes it considerably more difficult to replace the transponder.

Often only small wall thicknesses are available to accommodate a transponder, for example a glass tag protected by a housing, in or on a medical product. This means that classic snap-on elements such as snap hooks can hardly be realized due to their necessary dimensions. In addition, certain instruments such as motor-driven saws are exposed to high vibrations during operation, meaning that these snap-fit elements in small sizes are too delicate and can break in the long term.

### SUMMARY

Accordingly, the object of the present disclosure is to eliminate or at least reduce the disadvantages of the prior art.

More specifically, the object of the present disclosure is to provide a way of exchangeably connecting a transponder, which may include an individual identifier of a medical tool or instrument, to the medical tool or instrument in a manner protected from environmental influences and mechanical force without changing a geometry of the instrument or tool in such a way that handling properties of the instrument or tool are adversely affected.

Specifically, the object is solved by a protective housing for accommodating a transponder, which is receivable in a transponder receptacle formed therein, in a medical product, in particular in a medical instrument and/or medicine product in a sterilization cycle, wherein the protective housing includes/forms a form-fitting coupling device/site in the form of a circumferential groove/protective-housing groove for receiving a connecting spring and a connecting spring.

In other words, the object is solved by
a (separate) protective housing which is provided and configured to become/to be (permanently) installed with a medical product/tool and which for this purpose has or forms (integrally) a bearing device, preferably in the form of at least one protective-housing groove or latching notch, at least one projection or a similar form-fitting coupling device/point, which is provided and configured to come into or to be in bearing engagement with the medical product/tool, and
a transponder receptacle/transponder holder/sheathing which is formed/configured/provided in or by the protective housing and which is provided and configured such that the wirelessly readable transponder is inserted or insertable therein.

For example, the protective housing may be configured around the wirelessly readable transponder in such a way that the protective housing completely encloses the transponder or the transponder receptacle, in particular monolithically. Alternatively, the protective housing may contain an opening through which the transponder is insertable into the transponder receptacle. The transponder is in particular a passive RFID tag of the glass tag design. However, other types of transponder that may be read wirelessly, such as an NFC tag, are also conceivable. The protective housing contains the circumferential protective-housing groove on an outer circumferential wall. In other words, the protective-housing groove extends circumferentially all the way around/annularly around the protective housing and forms a channel-shaped recess in the outer circumferential wall of the protective housing. The protective-housing groove is provided and configured to receive the connecting spring.

By forming the protective housing with the protective-housing groove, the protective housing may be reversibly connected to the medical tool or instrument by the connecting spring. The connecting spring can absorb vibrations that occur during operation of the tool, so that damage to the transponder may be prevented. The protective housing may also prevent mechanical, thermal and chemical damage to the transponder.

In a first aspect, the protective-housing groove may be configured in a plane with or parallel to a central axis of the transponder receptacle.

In other words, the transponder receptacle may be a cylindrical recess. The transponder receptacle has the central axis. The central axis of the transponder receptacle and the protective-housing groove may be arranged in one plane. Alternatively, the central axis of the transponder receptacle may be configured parallel to the plane in which the protective-housing groove is arranged.

Such an arrangement of protective-housing groove and transponder receptacle may ensure that the transponder located in the transponder receptacle is integrated into the tool in such a way that the transponder is protected by the tool.

In a further aspect, the protective housing may have a substantially pill-shaped geometry, wherein the protective-housing groove extends in parallel wall portions of the pill-shaped geometry.

In other words, the protective-housing groove extends substantially along the protective housing.

In a further aspect, the parallel wall portions may have a first extension in a height direction normal to the plane with or parallel to the central axis of the protective housing and the protective housing may have a second extension in the height direction of the protective housing, wherein the first extension substantially corresponds to the second extent.

In a further aspect, the protective housing may be configured substantially symmetrically to the plane with or parallel to the central axis.

Furthermore, the object of the present disclosure is solved by the medical product, in particular medical instrument and/or medicine product in a sterilization cycle, having a protective-housing receptacle which is provided and configured to receive the protective housing and the transponder received in the protective housing. The protective-housing receptacle includes a respective wall groove for receiving the connecting spring in at least two mutually parallel side walls of the protective-housing receptacle.

In other words, the medical product includes the protective-housing receptacle, which is configured with the two side walls parallel to each other. One wall groove is configured in each of the side walls. The wall groove extends in particular over a full length/longitudinal extent of the parallel side wall. The wall groove extends in the depth direction away from a receptacle space of the protective-housing receptacle in a direction normal to the side wall. A depth of the wall groove in the depth direction is constant over the length of the parallel side wall. The wall groove is provided and configured to receive the connecting spring in a form-fitting manner.

Such a protective housing receptacle in the medical product is simple and inexpensive to manufacture, easily accessible and may also be implemented as a retrofit solution for existing instruments.

In one aspect, the protective-housing receptacle may be configured as a through-opening in the medical product or a wall of the medical product.

In other words, the protective-housing receptacle may connect an upper side of the medical product or the wall of the medical product to the bottom of the medical product or the wall of the medical product.

The protective housing receptacle is configured in such a way that the protective housing may be easily inserted into and removed from the protective housing receptacle.

In a further aspect, the two mutually parallel side walls of the protective-housing receptacle may be connected to each other by radius portions, wherein the wall groove ends in the radius portions.

In other words, the extension of the wall groove in the depth direction in the radius portions, starting from the parallel side walls, may decrease and in particular become zero. The longitudinal extent of the side walls may extend between the radius portions.

Such a configuration of the wall groove may ensure that the connecting spring comes/is in form-fitting engagement with the protective-housing receptacle in areas of the parallel side walls and that the connecting spring does not come/is not in form-fitting engagement with the protective-housing receptacle of the tool in the radius portions, which considerably simplifies insertion and removal of the protective housing from the protective-housing receptacle.

In a further aspect, a first distance of the wall groove to the upper side of the medical product may substantially correspond to a second distance of the wall groove to the bottom side of the medical product or the wall of the medical product.

In other words, the wall groove is configured in a height direction in the center of the protective housing receptacle.

Furthermore, the object of the present disclosure is solved by the connecting spring for form-fitting fixation of a protective housing in a medical product, wherein the connecting spring has two legs and a front portion connecting the legs. The connecting spring is configured substantially in a U-shape.

In other words, the connecting spring is provided and configured to form-fittingly engage in the wall groove of the protective-housing receptacle. The connecting spring is simultaneously provided and configured to engage in the protective-housing groove of the protective housing. The connecting spring is configured in the shape of a clasp.

In one aspect, the legs of the connecting spring may have a convex shape.

In other words, the legs in conjunction with the front portion of the connecting spring may have a substantially oval or oval-part geometry, which first widens starting from the front portion and then tapers toward an opening of the connecting spring.

In a further aspect, the connecting spring may be configured from a metal, in particular from a spring steel.

The object of the present disclosure is further solved by a protective housing system comprising the protective housing according to one of the above aspects and the connecting spring according to one of the above aspects. The connecting spring embraces the protective housing, wherein the front portion of the connecting spring is completely or approximately completely receivable or received in the circumferential groove of the protective housing and the legs of the connecting spring protrude beyond the protective housing in an untensioned state.

In other words, in the assembled state, the connecting spring embraces the protective housing in such a way that the legs of the connecting spring extend substantially parallel to the longitudinal direction of extension of the protective housing. In the assembled state, the front portion of the connecting spring is accommodated in the circumferential protective-housing groove of the protective housing. Preferably, the front portion of the connecting spring is accommodated in the circumferential protective-housing groove of the protective housing in such a way that the front portion does not protrude beyond the geometry of the protective housing.

In one aspect, the legs of the connecting spring may be at least partially receivable in the circumferential protective-housing groove of the protective housing in the tensioned state.

In other words, the legs of the connecting spring may be arranged parallel to the protective-housing groove of the protective housing in such a way that when the connecting spring is tensioned, the legs of the connecting spring plunge into the protective-housing groove of the protective housing.

Such a configuration of the protective housing system can ensure that when the protective housing system is inserted into the medical product or into the protective housing receptacle of the medical product, the connecting spring does not jam, wedge or slip and the form-fit between the connecting spring, the protective housing and the medical product can be reliably achieved.

The object according to the disclosure is further solved by a medical product system, with the protective housing according to one of the above aspects, the medical product according to one of the above aspects, and the connecting spring according to one of the above aspects. In the assembled state, the connecting spring engages in a form-fitting manner in the circumferential protective-housing groove of the protective housing and at the same time in a form-fitting manner in the wall groove of the protective-housing receptacle and thus fixes the protective housing in the medical product.

In other words, in the assembled state, the connecting spring, in particular in the front portion, may engage in a form-fitting manner in the circumferential protective-housing groove of the protective housing. In the assembled state, the legs of the connecting spring may engage at least in the wall groove of the protective housing receptacle.

In one aspect, the medical product may include disassembly openings that connect the wall groove to an environment surrounding the medical product system such that tensioning of the connecting spring is enabled in an assembled state.

In other words, the medical product may contain disassembly openings directly adjacent to the protective housing receptacle. The disassembly openings are holes/penetrations that allow a special tool to be inserted into the wall groove. The special tool may be used to press the legs of the connecting spring into the circumferential groove of the protective housing by overcoming a spring force of the connecting spring, thereby releasing the form-fit between the connecting spring, wall groove and protective-housing groove. The protective housing with the transponder held in the protective housing may then be removed from the medical product in a non-destructive manner.

The disassembly openings may be positioned in any orientation relative to the wall groove, as long as the connecting spring can be tensioned through the disassembly openings.

Preferably, the disassembly openings may be configured in the upper side and/or the lower side of the medical product and extend normally away from the upper side and/or lower side.

Alternatively or additionally, the disassembly openings may be configured on the side of the tool, preferably in a surface which is essentially at right angles to the upper side and/or the lower side, and may extend parallel to the upper side and/or the lower side.

In a further aspect, the medical product and/or the protective housing may be configured from a hard, inelastic material.

### BRIEF DESCRIPTION OF THE DRAWINGS

FIG. 1 shows a tool according to the disclosure with a protective housing receptacle;

FIG. 2 shows the protective-housing receptacle of the tool according to the disclosure in a sectional view;

FIG. 3 shows the protective housing receptacle of the tool according to the disclosure and a protective housing with a glass tag;

FIG. 4 shows a spring;

FIG. 5 shows the protective housing with the spring;

FIG. 6 shows the protective housing with the spring mounted in the protective housing receptacle in a first embodiment;

FIG. 7 shows the protective housing with the spring mounted in the protective-housing receptacle in a second embodiment; and

FIG. 8 shows the protective housing with the spring mounted in the protective housing receptacle in a third embodiment.

### DETAILED DESCRIPTION

Embodiments of the present disclosure are described below based on the accompanying Figures.

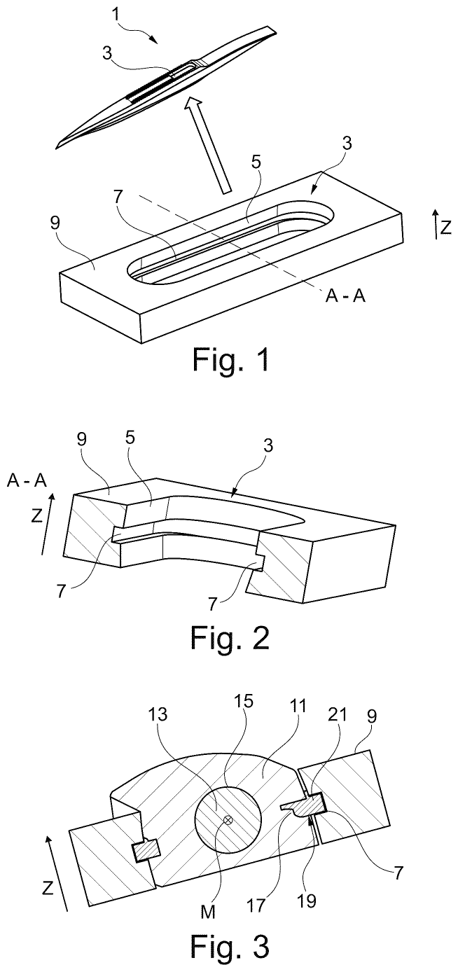

FIG. 1 shows a tool 1 according to the disclosure with a protective-housing receptacle 3. The protective-housing receptacle 3 is shown enlarged in FIG. 1. FIG. 2 shows the protective-housing receptacle 3 in a sectional view along line A-A. The protective-housing receptacle 3 forms a substantially cylindrical recess in the tool 1. A (negative-extruded) base surface of the protective-housing receptacle 3 is pill-shaped with two parallel portions and two radius portions delimiting the parallel portions. However, other geometries are also conceivable, wherein the geometry of the protective-housing receptacle 3 is matched to a geometry of the protective housing (see, for example, FIG. 6). An inner wall 5 of the protective-housing receptacle 3 contains a wall groove 7. The wall groove 7 is configured in the parallel portions of the protective-housing receptacle 3 and ends in the radius portions of the inner wall 5 of the protective-housing receptacle 3. However, embodiments are also conceivable in which the wall groove 7 is configured completely circumferentially in the inner wall 5 of the protective-housing receptacle 3. The wall groove 7 is configured as a substantially rectangular recess in the inner wall 5, which extends away from the protective-housing receptacle 3. The wall groove 7 is oriented parallel to a tool surface 9 and substantially centrally arranged/configured in a height direction Z in the protective-housing receptacle 3.

In the embodiment shown here, the protective-housing receptacle 3 is configured as a through-opening in the tool 1. However, embodiments in which the protective-housing receptacle 3 is configured as a kind of blind hole are also conceivable.

FIG. 3 shows the protective-housing receptacle 3 in a further sectional view, wherein in FIG. 3 a protective housing 11 with a glass tag 13, which is received in a tag receptacle 15 of the protective housing 11, is arranged in the protective-housing receptacle 3. The glass tag 13 is a wirelessly readable transponder element that allows at least one identification of the tool 1. Furthermore, tool-relevant information may be stored on the glass tag 13. The glass tag 13 may be completely surrounded by the protective housing 11. Alternatively, the tag receptacle 15 may be configured with an externally accessible opening that allows the glass tag 13 to be inserted into the protective housing.

The tag receptacle 15 extends in a longitudinal direction of the protective housing 11. The protective housing 11 also includes a protective-housing groove 17, which is configured circumferentially in an outer surface of the protective housing 11. The protective-housing groove 17 is configured in a plane with a central axis M of the tag receptacle 15. The wall groove 7 of the protective-housing receptacle 3 and the protective-housing groove 17 together form a groove space 19 in an assembled state. A spring 21 is configured in the groove space 19 to fix the protective housing 11 in the protective-housing receptacle 3 in a form-fitting manner. The protective housing 11 and the protective-housing receptacle 3 are matched to each other in such a way that the protective housing 11 is substantially completely accommodated in the protective-housing receptacle 3.

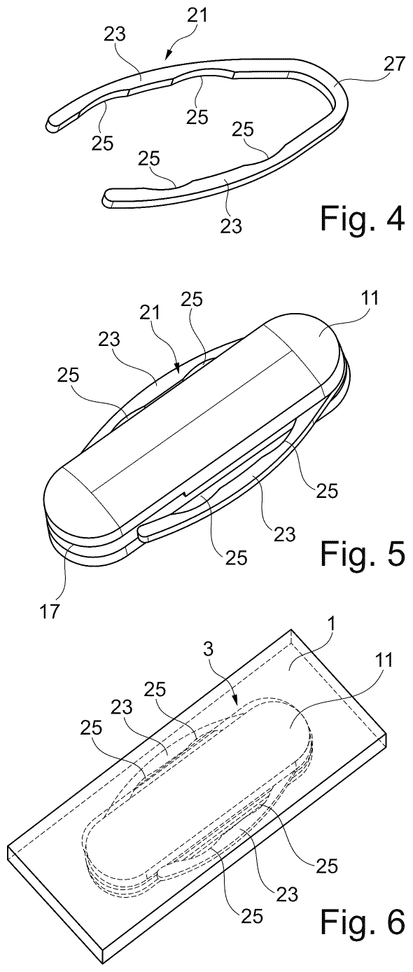

FIG. 4 shows the spring 21, which has a substantially U-shaped geometry. The spring 21 is configured in one piece from a spring steel and forms a clasp with two oval configured legs 23, which are open on one side. The legs 23 contain spherical recesses 25 on the inner sections facing each other. The two legs 23 are connected to each other at a front portion 27.

An assembly process of the protective housing 11 with the spring 21 in the protective housing receptacle 3 is described in more detail below with reference to FIG. 5 and FIG. 6.

FIG. 5 shows the protective housing 11 and the spring 21 in a pre-assembled state. In other words, the spring 21 is inserted into the protective-housing groove 17 of the protective housing 11. The legs 23 of the spring 21 protrude beyond the pill-shaped basic geometry of the protective housing 11, while the front portion 27 is completely accommodated in the protective-housing groove 17.

In order to fix the protective housing 11, which contains the glass tag 13, in the protective housing receptacle 3 configured in the tool 1, the legs 23 of the spring 21 are pressed together so that they are at least partially inserted into the protective-housing groove 17. The spring 21 is then tensioned. The protective housing 11 with the tensioned spring 21 is pushed into the protective-housing receptacle 3 by a user, wherein the inner wall 5 of the protective-housing receptacle 3 holds the spring 21 tensioned. When the protective housing 11 is in the position provided for the protective housing 11 in the protective-housing receptacle 3, i.e. the protective-housing groove 17 and the wall groove 7 of the protective-housing receptacle 3 are arranged in one plane, the spring 21 may relax and the legs 23 of the spring 21 engage in the wall groove 7 of the protective-housing receptacle 3. The spring 21 is then accommodated both in the wall groove 7 of the protective-housing receptacle 3 and in the protective-housing groove 17 and connects the protective housing 11 to the tool 1 in a form-fitting manner.

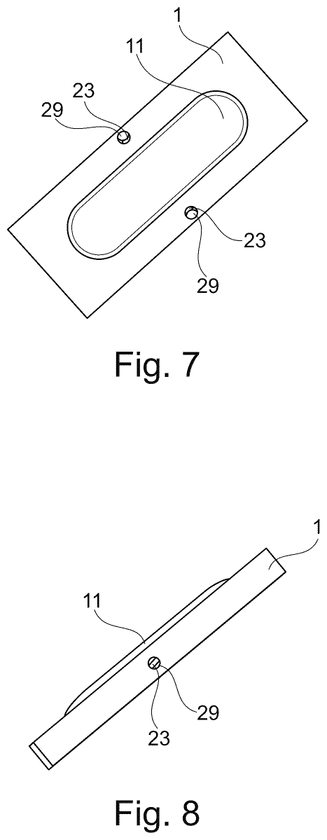

FIG. 7 shows the protective housing 11 with the spring 21 mounted in the protective housing receptacle 3 in a first alternative embodiment. This embodiment corresponds substantially to the embodiment described so far. In addition, the first alternative embodiment includes two disassembly openings 29. In the first alternative embodiment, the disassembly openings 29 are configured as holes that normally intersect/penetrate the wall groove 7 of the protective-housing receptacle 3. The disassembly openings 29 allow the protective housing 11 to be removed from the protective-housing receptacle 3. Specifically, the legs 23 of the spring 21 may be pressed together through the disassembly openings 29 using a special tool (not shown), thereby tensioning the spring 21 in such a way that the spring 21 and the wall groove 7 of the protective-housing receptacle 3 are disengaged. The protective housing 11 may then be removed from the protective housing receptacle 3.

FIG. 8 shows the protective housing 11 with the spring 21 mounted in the protective-housing receptacle 3 in a second alternative embodiment. This corresponds substantially to the first alternative embodiment. In contrast to the first alternative embodiment, the disassembly openings 29 are configured in one plane with the wall groove 7 and the protective-housing groove 17.

The arrangement of the disassembly openings 29 is primarily determined by the geometry of the tool 1.

In the embodiments shown here, the wall groove 7 is configured as a rectangular recess. Alternatively, the wall groove 7 may also be configured as a round or spherical or oval recess. In particular, if the wall groove 7 is configured as a round recess, the spring 21 may be configured from a round wire. With such a configuration of wall groove 7 and spring 21, a release force of the groove-spring connection may be set by selecting the overlap of the spring 21 and the wall groove 7. With a correspondingly low release force, tool-free disassembly may be implemented. With a complete overlap of the spring 21 and the wall groove 7, a non-detachable connection or a connection that cannot be detached without a tool may also be produced with this type of design.

In the embodiments shown here, the protective-housing groove 17 is configured in a plane with the central axis M of the tag receptacle 15. However, alternative embodiments are also conceivable in which the protective-housing groove 17 is not configured in a plane with the central axis M of the tag receptacle 15.

In the embodiments shown here, the spring 21 or the legs 23 of the spring 21 are configured with spherical recesses 25. These recesses 25 are not absolutely necessary. If a suitable material is selected for the spring 21, the recesses 25 may be dispensed with. The recesses 25 may be used to adjust the spring force of the spring 21 in particular.

### List of Reference Signs

1 tool
3 protective-housing receptacle
5 inner wall
7 wall groove
9 tool surface
11 protective housing
13 glass tag
15 tag receptacle
17 protective-housing groove
19 groove space
21 spring/connecting spring
23 legs
25 recess
27 front portion
29 disassembly opening
Z height direction
M central axis

1-14. (canceled)

15. A protective housing for a transponder, the protective housing being receivable in a medical product and comprising:
an outer surface that forms a perimeter;
a protective-housing groove extending along the perimeter;
a form-fitting coupling device configured to couple the protective housing to the medical product; and
a transponder receptacle for accommodating a transponder,
the form-fitting coupling device comprising a connecting spring receivable in the protective-housing groove.

16. The protective housing according to claim 15, wherein the protective-housing groove is configured in a plane with or parallel to a central axis of the transponder receptacle.

17. The protective housing according to claim 16, wherein the protective housing has a pill-shaped geometry.

18. The protective housing according to claim 17, wherein the protective-housing groove extends in parallel wall portions of the pill-shaped geometry.

19. The protective housing according to claim 18, wherein:
the parallel wall portions have a first extension in a height direction,
the protective housing has a second extension in the height direction,
the first extension corresponds to the second extension, and
the height direction is normal to the plane with or parallel to the central axis of the transponder receptacle.

20. The protective housing according to claim 18, wherein the protective housing is configured symmetrically relative to the plane with or parallel to the central axis of the transponder receptacle.

21. The protective housing according to claim 18, wherein the connecting spring comprises a front portion and two legs that connect with the front portion.

22. The protective housing according to claim 21, wherein:
the connecting spring embraces the protective housing,
the front portion of the connecting spring is completely receivable in the protective-housing groove, and
the two legs of the connecting spring protrude beyond the outer surface of the protective housing in an untensioned state.

23. The protective housing according to claim 22, wherein the two legs of the connecting spring are at least partially receivable in the protective-housing groove of the protective housing in a tensioned state.

24. A medical product comprising a body portion defining a protective-housing receptacle, the protective-housing receptacle having two mutually parallel side walls that define a wall groove for form-fitting and/or force-fitting receiving of a connecting spring, the protective-housing receptacle configured to receive a protective housing containing a transponder.

25. The medical product according to claim 24, wherein the protective-housing receptacle is configured as a through-opening in the medical product.

26. The medical product according to claim 24, wherein the two mutually parallel side walls of the protective-housing receptacle are connected to each other by radius portions, wherein the wall groove ends in the radius portions.

27. A connecting spring configured for form-fitting fixing of a protective housing in a medical product, the connecting spring comprising:
two legs; and
a front portion connecting the two legs,
the connecting spring being configured in a U-shape.

28. The connecting spring according to claim 27, wherein the two legs have a convex shape.

29. The connecting spring according to claim 27, wherein:
the two legs define mutually facing inner edges, and
each mutually facing inner edge comprises indentations spaced apart in a longitudinal direction of the two legs,
the indentations each reducing a width of each leg.

30. A medical product system comprising:
A. a protective housing for a transponder comprising:
i.) an outer surface that forms a perimeter;
ii.) a protective-housing groove extending along the perimeter;
iii.) a connecting spring receivable in the protective-housing groove; and
iv.) a transponder receptacle for accommodating the transponder,
B. a medical product defining a protective-housing receptacle, the protective-housing receptacle configured to receive the protective housing for the transponder, the protective-housing receptacle having two mutually parallel side walls that define a wall groove for form-fitting and/or force-fitting receiving of the connecting spring.

31. The medical product system according to claim 30, wherein, in an assembled state, the connecting spring form-fittingly engages in the protective-housing groove of the protective housing and simultaneously form-fittingly engages in the wall groove of the protective-housing receptacle to fix the protective housing in the medical product.

32. The medical product system according to claim 30, wherein the medical product further comprises disassembly openings that connect the wall groove to an environment surrounding the medical product system such that tensioning of the connecting spring is possible in an assembled state.
